(19) United States
(12) Patent Application Publication
(10) Pub. No.: US 2006/0029555 A1
(43) Pub. Date: Feb. 9, 2006

(54) Compositions and methods for prevention of photoaging

(76) Inventor: Eric F Bernstein, GLADWYNE, PA (US)

Correspondence Address: LICATLA & TYRRELL P.C., 66 E. MAIN STREET, MARLTON, NJ, 08053, US

(21) Appl. No.: 10/533,353

(22) PCT Filed: Oct. 29, 2003
(86) PCT No.: PCT/US03/34250

§ 371 Date: Jun. 16, 2005

Related U.S. Application Data
(60) Provisional application No. 60/423,409, filed on Oct. 31, 2002.

(51) Int. Cl. A61K 31/522; A61K 8/49

(52) U.S. Cl. 424/59; 514/263.32

(57) ABSTRACT

Methods of preventing photoaging and sun burn by topically applying a composition containing caffeine or a compound structurally similar to caffeine are provided. Pharmaceutical compositions comprising caffeine or a compound structurally similar to caffeine for the prevention of photoaging and sun burn are also provided.

### BACKGROUND OF THE INVENTION

The effects of ultraviolet radiation from exposure to the sun on human skin are a growing concern for today's longer-lived population. The majority of changes associated with an aged appearance result from chronic sun-damage (Warren et al., J. Am. Acad. Dermatol., 1991, 25:751-760; Frances, C. and Robert, L., Int. J. Dermatol., 1984, 23:166-179). Dramatic alterations of the superficial dermis accompany the deep wrinkles and laxity common in photoaged skin. The major histopathologic alteration of photoaged skin is the accumulation of material which, on routine histopathologic examination, has the staining characteristics of elastin and is, thus, termed solar elastosis. Immunohistochemical staining has shown the poorly-formed fibers comprising solar elastosis to be composed of elastin (Chen et al., J. Invest. Dermatol., 1986, 87:334-337; Mera et al., Br. J. Dermatol., 1987, 117:21-27) fibrillin (Chen et al., J. Invest. Dermatol., 1986, 87:334-337; Dahlback et al., J. Invest. Dermatol., 1990, 94:284-291; Bernstein et al., J. Invest. Dermatol., 1994, 103:182-186) and versican, the normal components of elastic fibers (Zimmerman et al., J. Cell. Biol., 1994, 124:817-825). A coordinate increase in elastin, fibrillin and versican mRNAs has been demonstrated in fibroblasts derived from photodamaged skin, as compared to fibroblasts derived from normal skin from the same individuals (Bernstein et al., J. Invest. Dermatol., 1994, 103:182-186). Elevated elastin mRNA levels in sun-damaged skin result from enhanced elastin promoter activity, as shown by transient transfections of fibroblasts with a DNA construct composed of the human elastin promoter linked to the chloramphenicol acetyltransferase (CAT) reporter gene (Bernstein et al., J. Invest. Dermatol., 1994, 103:182-186).

It has now believed that topical application of a composition comprising caffeine or a structurally related compound prevents photoaging and other skin damage resulting from exposure to solar, and more specifically, ultraviolet radiation.

### SUMMARY OF THE INVENTION

In the present invention, a new use is provided for compositions comprising caffeine or structurally related compounds. It is now believed that topical application of caffeine or a structurally related compound will provide protection against photoaging and other sun-damage such as sunburn caused by solar radiation. Accordingly, caffeine and compounds structurally similar to caffeine are believed to be useful as sunscreen agents. Compositions for use as sunscreen agents comprising caffeine or a compound structurally similar to caffeine are also provided.

### DETAILED DESCRIPTION OF THE INVENTION

Profound changes take place in the superficial dermis as a result of chronic sun-exposure. The major alteration is the deposition of massive amounts of abnormal elastic material, termed solar elastosis. It has been shown that solar elastosis is accompanied by elevations in elastin and fibrillin mRNAs and elastin promoter activity.

A transgenic mouse model which contains the human elastin promoter linked to a chloramphenicol acetyltransferase (CAT) reporter gene for testing compounds that may inhibit cutaneous photodamage has been developed. These mice express human elastin promoter activity in a tissue-specific and developmentally regulated manner. Promoter activity can be studied in this model as a function of small increases in ultraviolet radiation, demonstrating the sensitivity of the assay. In addition, quantitative data can be obtained after only a single exposure to ultraviolet radiation. A test compound is applied to the skin of a transgenic mouse capable of expressing the human elastin promoter. The transgenic mouse is then exposed to solar radiation and human elastin promoter activity in the mouse is determined. The human elastin promoter activity is then compared to that in transgenic mice also exposed to an equivalent dose of solar radiation which were not treated with the test compound to determine whether or not the test compound provided protection against the solar radiation. Since elastin promoter activation is a primary event in cutaneous aging, these mice represent a mouse model of human photoaging.

Using this transgenic mouse line, the ability of caffeine and compounds structurally similar to caffeine to inhibit the effects of solar radiation on human elastin promoter activity can be determined. In these experiments, mice will be divided into three groups, one group receiving no treatment, one group wherein a solution or suspension of caffeine or a compound structurally similar to caffeine in a pharmaceutically acceptable vehicle for topical application is applied topically to their backs, and a third group wherein the pharmaceutically acceptable vehicle alone is applied topically to their backs. Approximately fifteen minutes after topical application, the mice are exposed to 20 human minimal erythema doses (MEDs) of solar simulating radiation (SSR). Following phototreatment, the backs of the mice are rinsed twice with 70% isopropyl alcohol pads to remove any excess caffeine or compound structurally similar to caffeine. This procedure is repeated over three consecutive days.

Mice are then sacrificed and skin harvested for determination of CAT activity 24 hours after the third phototreatment. The baseline CAT activity of control mice receiving neither radiation nor treatment is standardized to a value of one. Relative increases in CAT activity in mice treated with vehicle alone are then compared with CAT activity in mice treated with vehicle containing caffeine or a compound structurally similar to caffeine.

Results of these experiments are expected to demonstrate that topical application of a composition comprising caffeine or a compound structurally related thereto to the skin provides protection against photoaging and other sun-damage such as sunburn. By “compound structurally similar to caffeine”, it is meant it is meant a compound with a similar chemical formula and structure which exhibits similar photodamage protective properties to caffeine. Examples include, but are not limited to, additional xanthines such as methylated xanthines theophylline and theobromine. Methods of rationally designing additional chemical compounds with similar structure to a known compound are well established and used routinely by those of skill in the art. Accordingly, upon reading of the instant application, structurally similar compounds to caffeine and other methylxanthines such as theophylline and theobromine for use in the present invention can be identified routinely by those of skill in the art.

Examples of compositions comprising caffeine or a structurally similar compound to caffeine include, but are not limited to creams, lotions and sprays. Methods of formulating caffeine of structurally similar compound to caffeine into creams, lotions and sprays as well as pharmaceutical additives for such formulations are well known to those skilled in the art. As will be obvious to those skilled in the art upon this disclosure, such compositions may further comprise secondary or additional sunscreens or free radical scavengers such as, but not limited to, Vitamin C and Vitamin E and analogs thereof. In a preferred embodiment, a composition comprising caffeine or a structurally similar compound to caffeine is applied to the skin prior to exposure to the sun. However, application of these compositions subsequent to the exposure can also mitigate any damage resulting to the skin from this exposure. It is believed that these compositions of the present invention will be especially useful in protecting individuals with heightened sensitivities to the sun, such as, but not limited to, individuals undergoing psoralen treatment for cancer, psoriasis and other skin conditions; individuals undergoing photodynamic therapy for skin cancer, psoriasis and other skin conditions; individuals suffering from genetic repair defects such as xeroderma pigmentosa, albinism or other conditions resulting from decreased endogenous melanin pigment.

The following nonlimiting examples are provided to further illustrate the present invention.

### EXAMPLES

### Example 1

### Transgenic Mice Expressing the Human Elastin Promoter

A homozygous line of transgenic mice expressing the 5.2-kb human elastin promoter linked to a CAT reporter gene is used. Hsu-Wong et al., J. Biol. Chem., 1994, 269:18072-18075. These mice express the human elastin promoter in a tissue-specific and developmentally regulated manner. Mice four or five days old were used since at this age, visible hair growth is not yet present.

### Example 2

### Solar Simulating Radiation

A Multiport Solar Simulator (Solar Light Company, Philadelphia, Pa.) containing a xenon arc lamp filtered through a Schott WG 320 filter (Schott Glaswerke, Mainz, Germany) can be used to administer solar simulating radiation (SSR). The output of the solar simulator is measured by means of a 3D UV meter (Solar Light Company) and displayed as human minimal erythema doses (MEDs). The emission spectrum of the lamp closely simulates solar radiation reaching the earth's surface. The light guides from the solar simulator are placed in light contact with the dorsal surface of the mice, which are restrained to prevent movement while SSR is administered.

Unirradiated control mice are also restrained without receiving SSR.

### Example 3

### CAT Assay

To measure the expression of the human elastin promoter/CAT reporter gene construct in the skin of transgenic mice and in fibroblast cultures established from these animals, CAT activity is determined. For extraction of the CAT from skin, the specimens are homogenized in 0.25 Tris-HCl, OH 7.5, using a tissue homogenizer (Brinkmann Instruments, Inc. Westbury, NY). The homogenates are centrifuged at 10,000 ×g for 15 minutes at 4° C. and the protein concentration in the supernatant determined by a commercial protein assay kit (Bio-Rad Laboratories, Richmond, Calif.). Aliquots of the supernatant containing 100 μg of protein are used for assay of CAT activity by incubation with [⁴C] chloramphenicol in accordance with well-known procedures. The acetylated and non-acetylated forms of radioactive chloramphenicol are separated by thin-layer chromatography and CAT activity is determined by the radioactivity in the acetylated forms as a percent of the total radioactivity in each sample.

What is claimed:

1. A method of protecting humans exposed to sunlight against photoaging and sunburn comprising-topically applying to skin of a human a composition comprising caffeine or a compound structurally similar to caffeine in an amount effective to protect the skin against photoaging and sunburn.

2. The method of claim 1 wherein the composition comprises caffeine, theophylline or theobromine.

3. The method of claim 1 wherein the composition is applied prior to exposure of the skin to sunlight.

4. The method of claim 1 wherein the composition is applied subsequent to exposure of the skin to sunlight.

5. A method of protecting individuals with a heightened sensitivity to the sun from damage resulting from the sun comprising topically applying to the skin of an individual with a heightened sensitivity to the sun a composition comprising caffeine or a compound structurally similar to caffeine prior to exposure of the individual to the sun.

6. The method of claim 5 wherein the composition comprises caffeine, theophylline or theobromine.

7. A pharmaceutical composition for prevention of photoaging and sunburn comprising caffeine or a compound structurally similar to caffeine and a second sunscreen or free radical scavenger.
